(19) United States
(12) Patent Application Publication
(10) Pub. No.: US 2024/0261765 A1
(43) Pub. Date: Aug. 8, 2024

(54) CONVERSION OF BETA-HYDROXY CARBONYL SPECIES AND PREPARATION OF AMINO ALCOHOL PRECURSOR USING BIFUNCTIONAL CATALYSTS DERIVED FROM LAYERED DOUBLE HYDROXIDES

(71) Applicant: Academia Sinica, Taipei City (TW)

(72) Inventors: PO-WEN CHUNG, Kaohsiung (TW); YUAN-PENG DU, Taipei (TW)

(21) Appl. No.: 18/429,813

(22) Filed: Feb. 1, 2024

Related U.S. Application Data
(60) Provisional application No. 63/442,532, filed on Feb. 1, 2023.

(51) Int. Cl. B01J 21/10; B01J 35/73; C07C 51/377

(52) U.S. Cl. CPC B01J 21/10; B01J 35/73; C07C 51/377; B01J 2523/22; B01J 2523/31; B01J 2531/847

(57) ABSTRACT

Disclosed are methods for conversion of β-hydroxy carbonyl species and preparation of amino alcohol precursor using bifunctional catalysts derived from layer double hydroxides. By the bifunctional catalyst, the abundant basic sites on HTO allow retro-aldol condensation to outpace direct hydrogenation, thus achieving an exceptional selectivity towards a desired product produced through retro-aldol condensation and then hydrogenation. Accordingly, this method exhibits particular utility in the renewable production of N-acetylethanolamine from biomass-derived N-acetyl glucosamine (GlcNAc) without using homogeneous base as a co-catalyst.

### CROSS REFERENCE TO RELATED APPLICATION

This application claims the benefit of filing date of U.S. Provisional Application Ser. No. 63/442,532 filed Feb. 1, 2023. The entirety of said Provisional Application is incorporated herein by reference.

### FIELD OF THE INVENTION

The present invention relates to conversion of β-hydroxy carbonyl species and preparation of amino alcohol precursor using bifunctional catalysts derived from layer double hydroxides.

### DESCRIPTION OF RELATED ART

Biomass is emerging as a promising renewable alternative to petroleum-based carbon resources due to the environmental impact caused by the production of petrochemicals from oil refineries. To date, research focuses have been mainly placed on the conversion of lignocellulosic biomass. In contrast, the valorization of marine biomass, that is the second most abundant biomass, has not been extensively studied. Marine biomass like crustacean shell is primarily composed of chitin, which copolymerizes the building units of glucosamine and N-acetylglucosamine (GleNAc). Thanks to the biologically fixed nitrogen in chitin's structure, marine biomass serves as a reservoir for not only renewable carbon but also nitrogen. Chitin and its monomers can be converted into many value-added chemicals, including N-containing furan derivatives, 5-hydroxymethylfurfural (HMF), protected amino acid, and acetic acid. Besides these chemicals, N-acetyl ethanolamine (NAEA) has gained an increased attention because ethanolamine (MEA, which can be produced by deacetylating NAEA) has numerous practical applications. For instance, ethanolamine is a feedstock in various industrial sectors (e.g., cosmetics and pharmaceuticals) and can be used for CO₂ capture. In this context, catalytic conversion of GlcNAc would be a key technology for producing sustainable NAEA from marine biomass. The production of NAEA from GlcNAc relied on a cascade reaction including retro-aldol condensation and subsequent hydrogenation of 2-acetamido-acetaldehyde intermediate (Scheme 1). However, achieving high NAEA yield is challenging because direct hydrogenation of GlcNAc is more favored than retro-aldol condensation in the presence of H₂, thus forming 2-acetamido-2-deoxysorbitol (r-GlcNAc) as the major product.

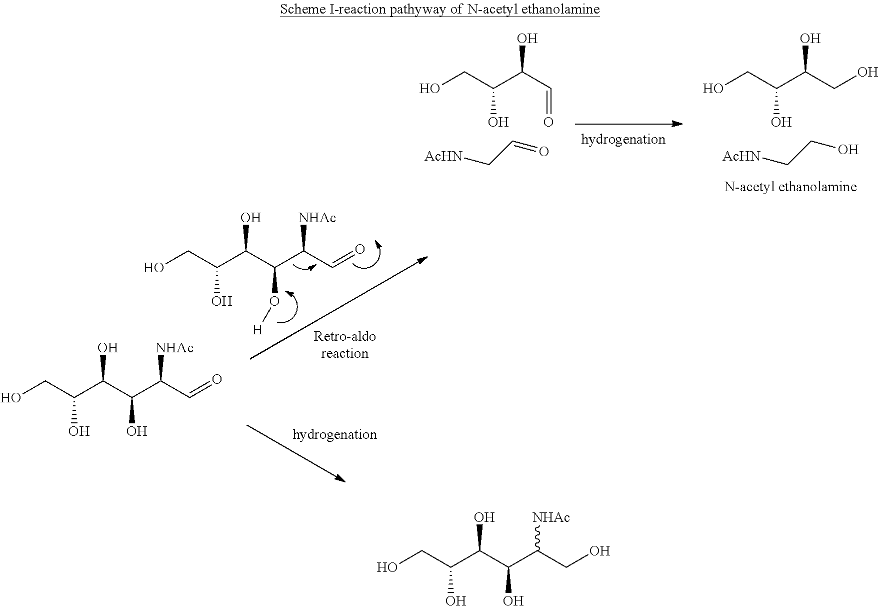

Bobbink et al. used NaOH and Ru/C to catalyze retro-aldol condensation and hydrogenation, respectively. An NMEA yield of 11% was obtained but uncleaved polyols were still the major products. Techikawara et. al. similarly used a metal-base (Ru/C—NaHCO₃) catalyst pair to facilitate the desirable cascade reaction. To further improve the NAEA yield, they lowered the H₂ pressure during the reaction to inhibit the rapid hydrogenation of GlcNAc and an increased yield of NAEA (15%) was obtained at 120° C. Both these prior arts relied on Ru-based catalyst and homogeneous base to produce NAEA from NAG. It would be challenging to produce NAEA using these processes in a large scale because of the use of costly Ru-based catalyst and the addition of homogeneous base catalyst, which requires further separation step for purifying products.

### SUMMARY OF THE INVENTION

An objective of the present invention is to provide a method applicable to synthesize NAEA from GlcNAc without using homogeneous base as a co-catalyst.

In accordance with the foregoing and other objectives, one aspect of the disclosure provides a composite catalyst, including a basic LDHs (layered double hydroxides)-derived mixed oxide support and hydrogenation-active species on the basic LDHs-derived mixed oxide support. The composite catalyst can be prepared by calcination of LDHs and then reduction under H₂ at a high temperature depending on the reducibility of the hydrogenation-active species, for example, at least about 700° C. for Ni, and at least about 500° C. for Cu. As the composite catalyst has both hydrogenation and base sites, retro-aldol condensation and subsequent hydrogenation of an intermediate produced from retro-aldol condensation can be catalyzed without necessitating the presence of a homogeneous base as a co-catalyst.

Another aspect of the disclosure provides a method for conversion of β-hydroxy carbonyl species, the method including: mixing β-hydroxy carbonyl species with the above-mentioned composite catalyst; and catalyzing retro-aldol condensation and then hydrogenation in hydrogen environment using the composite catalyst to complete conversion of the β-hydroxy carbonyl species. The β-hydroxy carbonyl species can be from biomass-derived feedstock, GlcNAc, and thus the disclosed method can be applied to produce an ethanolamine precursor (NAEA).

Yet another aspect of the disclosure provides a method for preparing an amino alcohol precursor, the method including: providing an amino saccharide; and mixing the amino saccharide with the above-mentioned composite catalyst for conversion of the amino saccharide into the amino alcohol precursor by retro-aldol condensation and then hydrogenation under catalysis of the composite catalyst. The step of providing the amino saccharide can include depolymerizing chitin biomass, and the weight ratio between the amino saccharide and the composite catalyst may be about 2 or less.

In the present invention, LDHs typically are doped M³⁺/N²⁺-LDHs, the M³⁺ is a trivalent metal, the N²⁺ is a bivalent metal, and the hydrogenation-active species are from doping elements of the doped M³⁺/N²⁺-LDHs. In one or more embodiments, the doped M³⁺/N²⁺-LDHs are Ni-doped hydrotalcite (HT) with the M³⁺ and N²⁺ being Al³⁺ and Mg²⁺, respectively, and the hydrogenation-active species are nickel nanoparticles. Accordingly, the earth-abundant composite catalyst (Ni, Al, and Mg) would be a highly favorable technology in circular economy. Herein, the total molar quantity of the doping elements and the N²⁺ can be about 2 to about 4 times that of the M³⁺. By calcination and then reduction, the Ni-doped HT can be converted into Ni/hydrotalcite oxide (HTO), represented by Ni/MgAlOₓ.

In the present invention, β-hydroxy carbonyl species may be one or more of β-hydroxyaldehyde and p-hydroxyketone, which can be from, for example, saccharide. The conversion of the β-hydroxy carbonyl species in the presence of the composite catalyst can be performed at a temperature in a range of about 60° C. to about 120° C. for about 1-4 hours. In one or more embodiments, the β-hydroxy carbonyl species includes a nitrogen-containing group at Cα position, and the conversion of the β-hydroxy carbonyl species allows for production of amino alcohol precursor. For example, the nitrogen-containing group may include N-acyl-substituted amino moiety, and the conversion can produce an alcohol compound containing an N-acyl-substituted amino moiety and having fewer carbon atoms than the β-hydroxy carbonyl species. The nitrogen atom of the nitrogen-containing group can be directly attached to the α-carbon of the β-hydroxy carbonyl species. In a particular example, the β-hydroxy carbonyl species is from an amino saccharide (such as GlcNAc), and the conversion involves first cleaving C2-C3 bond of GlcNAc by the basic support through retro-aldol condensation and then hydrogenating 2-acetamido-acetaldehyde intermediate by Ni nanoparticles, forming N-acetyl ethanolamine as the amino alcohol precursor (i.e. ethanolamine precursor). N-acetyl ethanolamine can be further deacetylated to form ethanolamine, which is an important bulk chemical that serves as an intermediate in various industrial sectors, including cosmetics, pharmaceuticals and CO₂ capture.

As used herein, the term “acyl” represents groups of 1, 2, 3, 4, 5, 6, 7, 8 or more carbon atoms of a straight, branched, cyclic configuration, saturated, unsaturated and aromatic, and combinations thereof, or hydrogen, attached to the parent molecular group through a carbonyl group. This group is exemplified by formyl (—C(O)H), acetyl (Ac or —C(O)Me), propionyl, isobutyryl, butanoyl, and the like.

As used herein, the term “high temperature” refers to temperatures greater than 300° C., and preferably greater than 400° C. to 1000° C., such as 500° C.-900° C., 600° C.-900° C., 700° C.-900° C., and 700° C.-800° C.

As used herein, the term “about” is understood to account for minor increases and/or decreases beyond a recited value, which changes do not significantly impact the desired function of the parameter beyond the recited value(s). In some cases, “about” encompasses +/−10% of any recited value. As used herein, this term modifies any recited value, range of values, or endpoints of one or more ranges.

As used herein, the term “LDH (layered double hydroxide)” refers to a class of materials with positively charged layers and weakly bound charge-balancing anions located in the interlayer region and having the structural memory effect property which allows destroyed layered structures to be reconstructed into rehydrated LDHs under certain circumstance. At moderate calcination temperature, about 300-600° C., the LDH can undergo thermal decomposition to metal oxide. The LDHs mentioned herein are not particularly limited, and may be any monometallic LDHs, multimetallic LDHs (e.g. binary, ternary, quaternary LDHs), metal-loaded LDHs or the like. The LDHs may be represented by the general formula: Mx3+N₍₁₋ₓ₎²⁺ (OH)₂Aⁿ⁻γH₂O, where M³⁺ and N²⁺ are trivalent and bivalent metal ions, respectively, and Aⁿ⁻ is the interlayer ion of valence n. The x value represents the proportion of trivalent metal ion to the proportion of total amount metal ion and y denotes variable amounts of interlayer water. Common forms of LDH include Mg²⁺ and Al³⁺ as predominant metal species in a lattice of the LDH (i.e. Al³⁺/Mg²⁺-LDH, known as hydrotalcite) and Mg²⁺ and Fe³⁺ (i.e. Fe³⁺/Mg²⁺-LDH, known as pyroaurites). Further, additional metals other than the predominant metal species may be incorporated in the LDH to form metal-loaded M³⁺/N²⁺-LDH (such as Ni-loaded HT Cu-loaded HT, Ru-loaded HT and other metal-loaded HT).

As used herein, the term “saccharide” refers to sugars or sugar derivatives, polyhydroxylated aldehydes and ketones with an empirical formula that approximates Cₘ(H₂O)ₙ, i.e., wherein m and n are the same or about the same integer. The term is not intended to be limited to any saccharides and encompasses monosaccharides, disaccharides, oligosaccharides, polysaccharides and derivatives thereof (e.g. N-acetyl-glucosamine, glucosamine and any other amino saccharides).

As used herein, the term “or” is used to link alternatives provided within a list. The use of this term does not exclude the use of such alternatives together, such as in a combination; and the use of this term does not indicate or require that an alternative must be used alone. The use of this term can indicate that the alternative can be used alone or can be used together with any other alternative within the list.

As used herein, the phrase “one or more of A, B, and C” should be construed to mean a logical (A OR B OR C), using a non-exclusive logical OR, and should not be construed to mean “one or more of A, one or more of B, and one or more of C.”

These and other features and advantages of the present invention will be further described and more readily apparent from the detailed description of the preferred embodiments which follows.

### BRIEF DESCRIPTION OF THE DRAWINGS

FIG. 1 shows a schematic depiction of the conversion of GlcNAc into NAEA using a composite catalyst including a basic MgAlOₓ support and Ni nanoparticles.

FIG. 2 shows X-ray diffractogram of Ni/HTO-2Mg (catalyst A), Ni/HTO-3Mg (catalyst B) and Ni/HTO-4Mg (catalyst C).

FIG. 3 shows TEM image and particle size distribution of Ni/HTO-2Mg (a), Ni/HTO-3Mg (b) and Ni/HTO-4Mg (c).

FIG. 4 shows X-ray diffractograms of Ni/HT-2Mg, Ni/HTO-2Mg-600 (reduced at 600° C.) and Ni/HTO-2Mg, in which the asterisk and cross signs denote the diffractions of metallic Ni and MgO, respectively.

FIG. 5 shows TPR profiles of NiO/HTO-nMg and NiO/MgO (a) and CO₂-TPD profiles of Ni/HTO-nMg and Ni/MgO (b).

FIG. 6 shows DRIFTS analysis of CO₂ chemisorbed on (a) Ni/HTO-2Mg (a) and Ni/MgO (b).

FIG. 7 shows catalytic results of GlcNAc conversion at 90° C. under 145 psi H2 for 1 h (a) and 4 h (b) using Ru/C—NaHCO₃ and bifunctional catalysts.

FIG. 8 shows NH₃-TPD results of Ni/HTO-nMg and Ni/MgO.

### DETAILED DESCRIPTION OF THE PREFERRED EMBODIMENTS

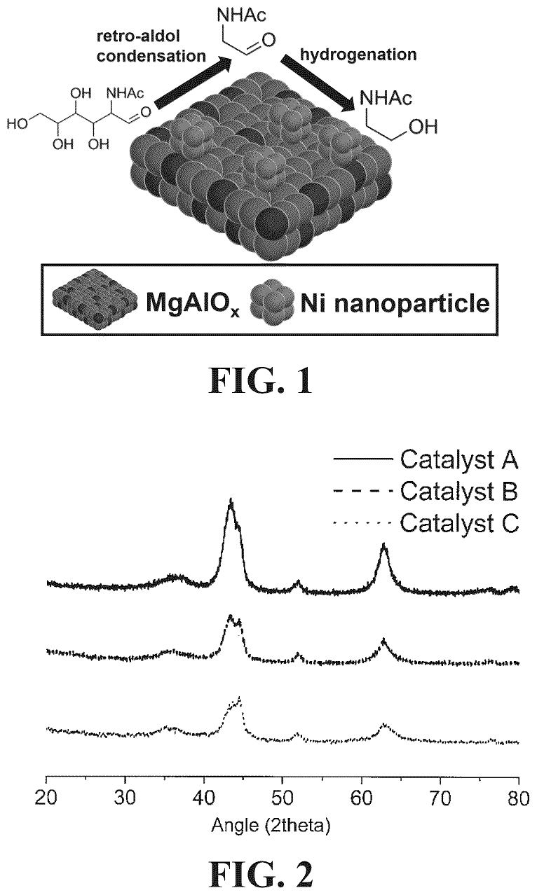

Described herein is a bifunctional solid catalyst, which possesses both hydrogenation and base sites. Specifically, LDHs are used as a precursor to synthesize hydrogenation-active species (such as Ni catalysts) supported on basic LDHs-derived mixed oxides (such as magnesium-aluminum mixed oxide, MgAlOₓ). The basic support enables base-catalyzed reactions to proceed on catalyst surface. This allows retro-aldol condensation prevails over direct hydrogenation at elevated temperature when surface basic sites outnumber hydrogenation sites. Accordingly, the LDHs-derived catalyst can be employed to produce NAEA from GlcNAc, as shown in FIG. 1. In addition to NAEA, other identified products, including ManNAc, r-GlcNA, r-ManNAc, and the diastereomers of 2-acetamido-2,3-dideoxyhexitol (expressed as deoxy-HexNAc), are formed during GlcNAc conversion through epimerization, direct hydrogentation, and dehydration, respectively (Scheme II).

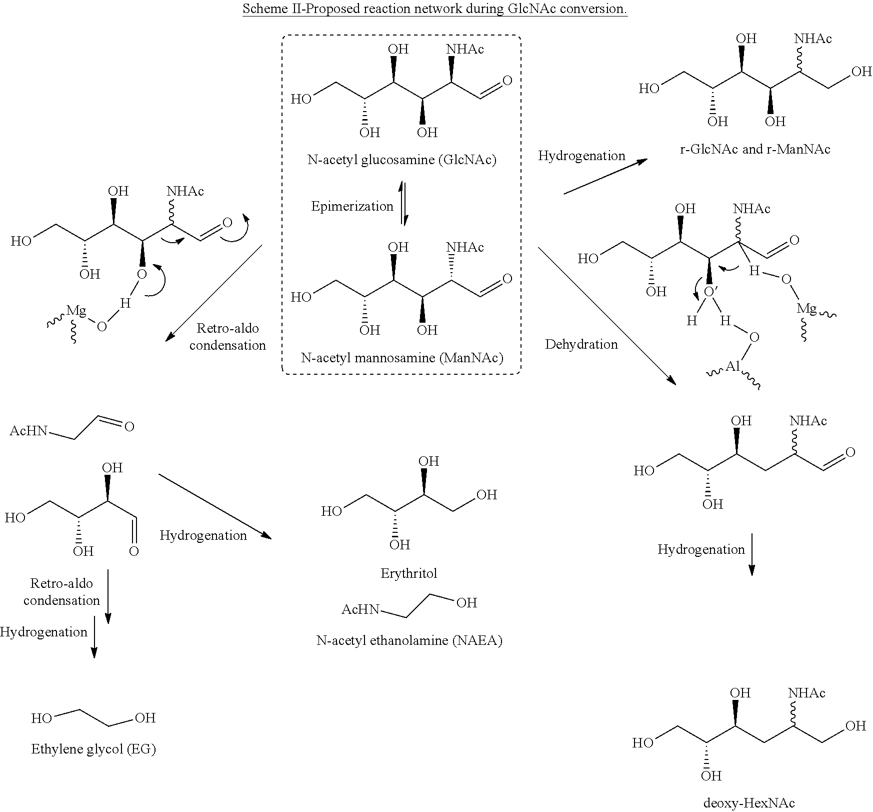

### Experimental Details

### Chemicals and Materials

Mg(NO₃)₂·6H₂O(Alfa-Aesar, 98-102%), Ni(NO₃)₂·6H₂O (Alfa-Aesar, 98%) Al(NO₃)₃·9H₂O (Fisher Scientific, 98%), Na₂CO₃ (Sigma-Aldrich, 99.8%), NaHCO₃ (Sigma-Aldrich, ACS grade), NaOH (Shimakyu's Pure Chemicals, 99.4%), methanol (Macron Fine Chemicals, 99.8%), KBr (Sigma-Aldrich, IR grade)N-acetyl glucosamine (Sigma-Aldrich, 99%), N-Acetyl mannosamine (TCI chemical, 97%), Ethylene glycol (Vetec, 98%), meso-erythritol (TCI chemical, 99%), N-Acetyl ethanolamine (Combi-Blocks, 95%) and Ru/C catalyst (TCI chemical, wetted with 50% H₂O) were purchased and used without further purification. The water used in this study was deionized using a Labconco WaterPro Plus station. All gases were purchased from Air Product.

### Synthesis of LDHs-Derived Catalysts

### [Catalysts A-C]

The bifunctional composite catalyst containing Ni particles as hydrogenation-active species on a basic support was derived from LDHs synthesized using co-precipitation method. The LDHs were hydrotalcite materials whose Mg was partially substituted with Ni by adding nickel nitrate during co-precipitation. The molar ratio of Mg+Ni/Al was varied from 2 to 4 and a series of Ni-doped hydrotalcite-like precursors (Ni/HT-nMg, where n denotes Mg+Ni/Al, ranging from 2 to 4) whose general formula is MgₓNi_{y}Al₁CO₃(OH)₁₆·4H₂O (wherein x+y=n) was prepared by coprecipitation at controlled pH.

Typically, A mixture of metal nitrate solution was prepared by dissolving Mg(NO₃)₂·6H₂O, Ni(NO₃)₂·6H₂O and Al(NO₃)₃·9H₂O in 200 mL in a solvent pair containing 100 mL methanol and 100 mL water (MeOH—H₂O). The concentrations of Mg²⁺, Ni²⁺, and Al³⁺ were 0.09 M, 0.01 M and 0.05 M, respectively. In parallel, an alkaline solution was prepared, which contains 0.2 M NaOH and 0.08 M Na₂CO₃, similarly in 200 mL MeOH—H₂O. Coprecipitation of Ni/HT-2Mg was proceeded by adding the metal nitrate solution into MeOH—H₂O at a rate of 2 mL min⁻¹ while regulating the pH value at 10 by adding the alkaline solution described above. Ni/HT-3Mg and Ni/HT-4Mg were prepared using the same procedure except the Al³⁺ concentrations were varied (0.033 M for Ni/HT-3Mg and 0.025 M for Ni/HT-4Mg). After coprecipitation, the slurry was aged in a sealed polypropylene bottle at 65° C. for 24 h in a conventional oven and the resulting precipitate was collected by filtration. The crude Ni/HT-nMg was thoroughly washed with deionized water until the pH of the filtrate is below 8. The resulting paste was dried at 105° C. for 15 h in a conventional oven. NiO/HTO-nMg was prepared by calcining the corresponding Ni/HT-nMg at 500° C. for 12 h with a heating ramp of 2° C. min⁻¹ in a muffle furnace. Prior to activity measurement, NiO/HTO-nMg was reduced at 700° C. for 5 h with a heating ramp of 2° C. min−1 in a tubular furnace under a continuous flow of H₂. The activated catalysts, Ni/HTO-nMg, are denoted as catalyst A (Ni/HTO-2Mg), catalyst B (Ni/HTO-3Mg) and catalyst C (Ni/HTO-4Mg).

### [Catalyst D]

The procedure as described earlier for catalyst A was repeated, except that Ni(NO₃)₂·6H₂O was replaced by Cu(NO₃)₂·6H₂O. The reduction temperature was set to 500° C. because of the better reducibility of Cu, which was confirmed by temperature programmed reduction analysis. The resulting catalyst is denoted as catalyst D.

### Synthesis of Comparative Catalysts

### [Catalyst E]

Ni/MgO was prepared using a modified method described above. In brief, a metal nitrate solution containing 0.015 M Ni²⁺ and 0.2 M Mg²⁺ was prepared by dissolving Mg(NO₃)₂·6H₂O and Ni(NO₃)₂·6H₂Oin MeOH—H₂O. Coprecipitation was performed by adding this nitrate solution into MeOH/H₂O at a rate of2 mL min⁻¹ while the pH value was controlled at 10 by using a 0.44 M NaOH in MeOH—H₂O. Then the aforementioned separation, washing and thermal treatment steps were repeated to obtain Ni/MgO, denoted as catalyst E.

### [Catalyst F]

The procedure as described earlier for catalyst A was repeated without Mg(NO₃)₂·6H₂O. The concentrations of Ni²⁺ and Al³⁺ were adjusted to 0.1 M and 0.033 M, respectively. The resulting catalyst is demoted as catalyst F.

### [Catalyst G]

The 1.23 g Ni(NO₃)₂·6H₂O was dissolved in 5 mL deionized water. WO₃ was mixed with this solution and dried using a rotary evaporator (wet impregnation), Then the same drying, calcination and reduction steps were repeated as catalyst A. The resulting catalyst is demoted as catalyst G.

### Catalyst Characterization

N₂ physisorption and H₂ chemisorption were conducted on a Micromeritics 3 Flex instrument. For N₂ physisorption, the samples were degassed and dried under reduced pressure at 150° C. prior to measurement. The adsorption and desorption of N₂ were performed at −196° C. cooled in liquid N₂. The specific surface area (S_{BET}) was determined using Brunauer-Emmett-Teller (BET) theory. For H₂ chemisorption, the samples were first reduced at 400° C. under H₂ for 1 h. Then the double isotherm method was performed, where the catalyst surface was first saturated by physisorbed and chemisorbed H₂ and then second adsorption was conducted after removing the physisorbed H₂ under reduced pressure. The amount of chemisorbed H₂ was calculated by the subtraction method of two adsorption isotherms.

Temperature programmed reduction (TPR) as well as temperature programmed desorption of CO₂ and NH₃ (CO₂-TPD and NH₃-TPD) were conducted on a Micromeritics AutoChem II 2920. The samples were dried under He flow (30 mL min⁻¹) at 500° C. for 2 h prior these analyses. TPR was performed under the flow of 10% H₂ (20 mL min⁻¹, balanced in Ar). The temperature was ramped (10° C. min⁻¹) from 50° C. to 800° C. while the consumption of H₂ was recorded by a thermal conductivity detector (TCD). For CO₂-TPD analysis, the sample was saturated with CO₂ by flowing 10% CO₂ (50 mL min⁻¹, balanced in He) for 1 h at 40° C. The weakly adsorbed CO₂ was purged by switching CO₂ to He and then He was continuously flowed 1 h. Subsequently, the temperature was ramped (10° C. min⁻¹) from 40° C. to 800° C. while the desorption of CO₂ was recorded by a TCD. For NH₃-TPD analysis, the sample was saturated with NH₃ by flowing 10% NH₃ (30 mL min⁻¹, balanced in He) for 1 h at 35° C. The purging and desorption steps were the same as those of CO₂-TPD.

Powder X-ray diffraction (PXRD) was conducted on a Bruker D8 Advance X-ray diffractometer operated at 40 kV and 40 mA. Cu Kα (λ=1.5406 Å) was used as a radiation source.

X-Ray Photoelectron Spectroscopy (XPS) measurements were carried out using a PHI QuanteraII instrument with a scanning X-ray microprobe (Al anode). The beam size and power were 200 μm and 45 W, respectively. The pass energy was set to 55 eV to probe Mg 2p, Al 2p and Ni 2p with a step size of 0.1 eV.

Nuclear Magnetic Resonance (NMR) was performed in a Bruker Avance 500 MHz spectrometer with a TXI (¹H/¹³C/¹⁵N) 5 mm CryoProbe with Z gradient.

The Ni particles of the catalysts were imaged using a high-resolution transmission electron microscopy (HR-TEM, JEM-2100F, JEOL, Japan) operated at 200 kV. The specimen was prepared by dispersing the catalyst suspension (in ethanol) onto a carbon-lacey Cu grid (300 mesh) and dried at ambient temperature. Ni particle size was determined by sampling 50 Ni particles that have high contrast with MgAlOₓ support.

Diffuse reflectance Fourier transform infrared spectroscopy (DRIFTS) with CO₂ probe was measured on a Jasco FTIR 6700 instrument equipped with a Harrick Praying Mantis accessory. Sample was prepared by thoroughly mixing catalyst with KBr (catalyst/KBr= 1/60) using a mortar and a pestle. The physisorbed water was removed by heating the sample to 400° C. under continuous N₂ flow (40 mL min⁻¹) for 1 h. Sample was cooled down to 35° C. and CO₂ was subsequently fed (40 mL min⁻¹) into the chamber for 30 min. The weakly adsorbed CO₂ was purged by switching CO₂ to N₂ and then N₂ flow was maintained for 30 min. The DRIFTS spectra were recorded at 35 and 200° C. under a continuous flow of N₂.

### Conversion of Amino Saccharide

The catalytic conversion of amino saccharide (GlcNAc) under reductive environment was performed in a Biotage Endeavor Pressure Reactor. Typically, 20 mg of catalyst was mixed with 4 mL GlcNAc(aq) (10 mg-mL⁻¹) in a glass tube. The solution was transferred into the pressure reactor. The headspace was purged and then the reactor vessel was pressurized with H₂ to 145 psi. The mixture was mechanically stirred with a turbine at 500 rpm and the reaction proceeded at a range between 60° C. and 90° C. After the reaction was completed, the solid catalyst and soluble products were separated by centrifugation and filtration. The calculations of the measures for evaluating catalytic activity were shown below. ManNAc is used as an example to demonstrate the calculation of yield and selectivity. Other products were calculated in a similar way.

$$Conversion ( X ) = \frac{G l c N A c ( {t}_{0} ) - G l c N A c ( t )}{G l c N A c ( {t}_{0} )} \times 100 \%$$

$${Yield}_{M a n N A c} ( C \% ) = \frac{C weight in ManNAc ( t )}{C weight in GlcNAc ( {t}_{0} )} \times 1 \; 00 \%$$

$${Selectivity}_{M a n N A c} = \frac{{Yield}_{M a n N A c}}{Conversion} \times 100 \%$$

Additionally, the conversion of GleNAc into NAEA was also taken at 120° C. to compare the yields of NAEA under Catalysts A-D and F-G.

### Product Analysis and Quantification

The products were analyzed using a high-performance liquid chromatography (HPLC) equipped with a differential refractive index detector (RID). The filtered product mixture was analyzed using a Biorad Aminex HPX-87H column (300 mm×7.8 mm, mobile phase: 0.01N H₂SO_{4(aq)}) with a flow rate of 0.6 mL min⁻¹ at 50° C. Because erythritol and GlcNAc are not resolved in HPX-87H column, GlcNAc in the product streams resulting from GlcNAc hydrogenation at 90° C. using Ni/HTO-Mg and Ni/MgO (in which erythritol yield is above 5%) was quantified by a Shodex Ligand Exchange SP0810 Column (300 mm×8.0 mm, mobile phase: H₂O) with a flow rate of 1 mL min⁻¹ at 70° C. GlcNAc, ManNAc, ethylene glycol, erythritol and NAEA were quantified using the peak areas generated by RID and calibration curves obtained from diluted analytical grade standards. For the quantification of erythritol, the unresolved peak was deconvoluted by subtracting the theoretical peak area of GlcNAc from the area of the overlapped peak. The theoretical peak area of GlcNAc was estimated by using its quantity (through analysis using Shodex SP0810 Column) and the calibration curve for HPX-87H column. The analytical grade standards of r-ManNAc, r-GlcNAc and HexNAc were not commercially available, so their calibration curves were produced using self-prepared standards and the preparation methods were described below.

Purification of r-ManNAc, r-GlcNAc and Deoxy-HexNAc for HPLC Calibration

The r-ManNAc and r-GlcNAc standards were prepared by hydrogenating GlcNAc and ManNAc, respectively using Ru/C at 90° C. for 1 h under 145 psi H₂. The mass ratio of catalyst and reactant was maintained at ½. Because Ru/C hydrogenates GlcNAc and ManNAc with 100% selectivity, the standards of r-ManNAc and r-GlcNAc can be obtained through lyophilizing the filtered product solution without further purification.

For deoxy-HexNAc, GlcNAc hydrogenation was performed at 90° C. for 4 h under 145 psi H₂ using Ni/HTO-2Mg (mass of catalyst/mass of GlcNAc=½). The fully reacted solution was lyophilized, resulting in a viscous liquid mixture. The mixture was redissolved in methanol and purified using silica gel column chromatography with methanol and chloroform (methanol/chloroform=½). The separated products were tracked through thin-layer chromatography (TLC) with pre-coated glass plates of silica gel 60 F254 (0.25 mm, E. Merck). The spots were visualized by dipping the TLC plate into Hanessian's Stain (cerium ammonium molybdate) and subsequently heating it on a hot plate. The purified product was dried under reduced pressure at room temperature and its major components were identified as the diastereomeric isomers of 2-Acetamido-2,3-dideoxyhexitols (deoxy-HexNAc), whose structures were confirmed using ¹³C and ¹H NMR spectroscopy as well as electrospray ionization mass spectrometry (ESI-MS). The second most abundant component is erythritol, which was identified through HPLC analysis by means of its known retention time. The rest of minor species only account for approximately 5% of the total RID peak area in the chromatograph. Accordingly, the actual weight of the self-prepared deoxy-HexNAc standard was determined by subtracting the erythritol weight (estimated using the peak area in the resulting chromatogram of the diluted standard) from the pre-measured weight and then multiply the resulting value by 0.95. A series of diluted deoxy-HexNAc standards was prepared and analyzed for obtaining the calibration curve of deoxy-HexNAc.

### Result and Discussion

### Catalyst Characterization

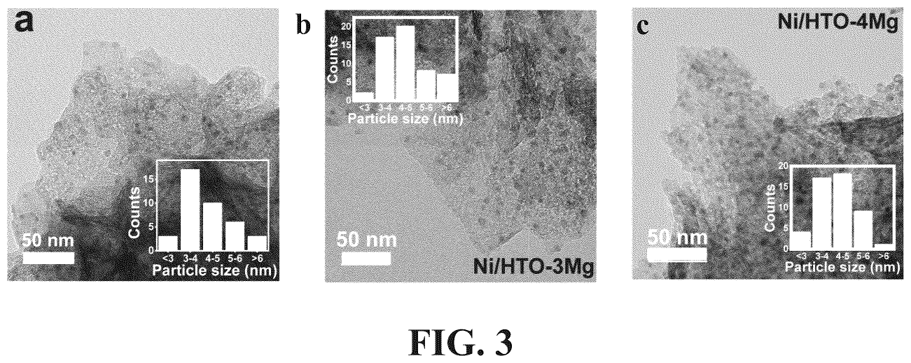

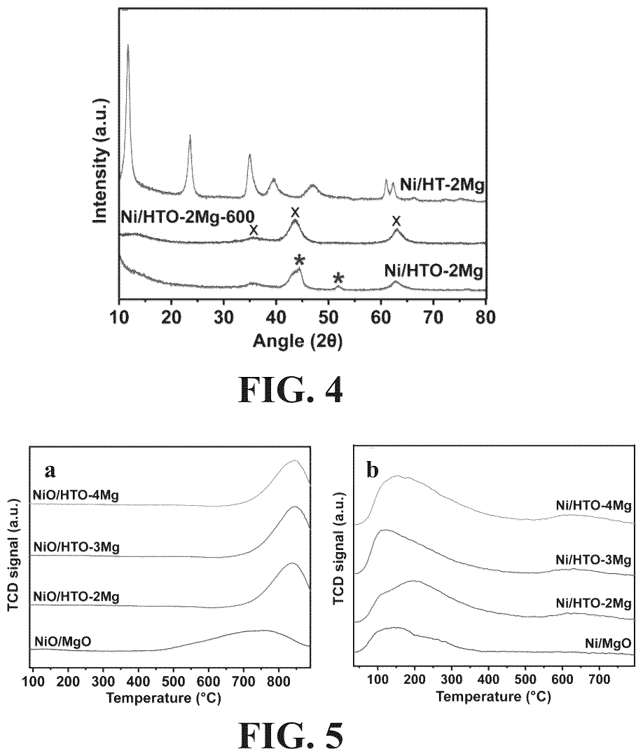

The calcined Ni/HT-nMg (NiO/HTO-nMg) were thermally reduced with continuous flow of H₂, resulting in Ni/HTO-nMg, which comprises Ni particles supported on MgAlOₓ (HTO). The appearance of X-ray diffraction at 44 degree indicates the formation of Ni nanoparticles after these thermal treatments (FIG. 2). TEM analyses showed that the Ni particle size of Ni/HTO-nMg ranges from 4 to 5 nm (FIG. 3; Ni/HTO-2Mg: 4.3±1.1 nm, Ni/HTO-3Mg: 4.6±1.1 nm, Ni/HTO-4Mg:4.2±0.9 nm). XRD analyses also suggested that thermal treatments transformed LDH structure into segregated phases containing Ni particles and MgAlOₓ (FIG. 4, uppermost and lowermost lines). Temperature-programmed reduction (TPR) profiles indicated that the onset of the reduction of NiO/HTO-nMg is around 700° C. (FIG. 5a). In comparison, the reduction of NiO/MgO (prepared similarly using co-precipitation method followed by calcination) began at 500° C. Agreeing with previous reports, Ni²⁺ was likely incorporated into LDH's crystal structure, thus lowering its reducibility. Indeed, when NiO/HTO-2Mg was reduced at 600° C., the diffraction peak of Ni was absent (FIG. 4, middle line) and the resulting catalyst showed a poor activity. Therefore, all the catalysts used in this study were reduced at 700° C. for 5 h prior to activity assessments.

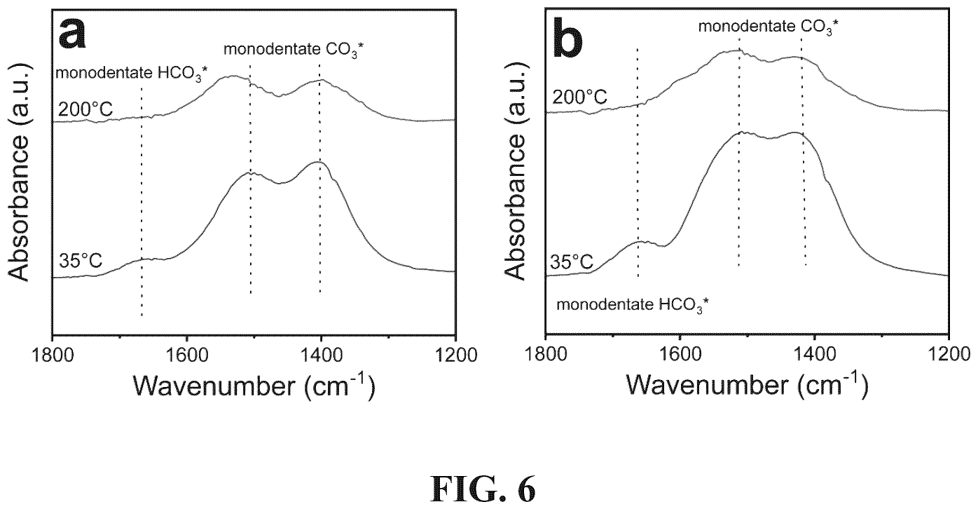

The surface basicity of Ni/HTO-nMg and Ni/MgO were analyzed and quantified by temperature-programmed desorption using CO₂ as a probe molecule (CO₂-TPD). All catalysts show a broad desorption band at the temperature range of 100 to 300° C. (FIG. 5b), which can be attributed to medium basic sites. For Ni/HTO-nMg, an additional desorption band can be observed at above 600° C., suggesting the existence of stronger basic sites. The absence of stronger basic site on Ni/MgO implied that the basicity of some Mg species might be enhanced when Mg coordinates with Al. To confirm that CO₂ chemisorb on basic sites, in-situ diffuse reflectance infrared Fourier transform spectroscopy (DRIFTS) was conducted (FIG. 6). After CO₂ adsorption, monodentate CO₃*and HCO₃*were found on both Ni/HTO-2Mg and Ni/MgO. It was reported that CO₃*resulted from CO₂ interacting with medium and strong basic sites (e.g., Mg 0 moiety). In comparison, HCO₃*formed on weaker basic sites (e.g., Mg OH moiety) and it desorbed at 200° C. These characterization results substantiate the surface biofunctionality of Ni/HTO-nMg and Ni/MgO whose hydrogenation and basic sites might enable the cascade reaction of GlcNAc.

The characterization results of Ni-based catalysts were summarized in below Table 1. The analyses of ICP-Mass, N₂ physisorption, XPS, static H₂ chemisorption, CO₂-TPD and NH₃-TPD showed Ni loading of about 6-10%, S_{BET} of about 200-250 m²/g, surface Mg/Al ratio of about 1-3, about 50-75 μmol/g, about 700-850 μmol/g and about 100-250 μmol/g, respectively.

TABLE 1

| Cata- | Loading | S_{BET} | Mg/Al | Chemisorbed | CO₂-TPD | NH₃-TPD |
|---|---|---|---|---|---|---|
| lyst | (Ni %) | (m²/g) | ratio | H₂ (μmol/g) | (μmol/g) | (μmol/g) |
| A | 6.9 | 216.6 | 1.7 | 52 | 731 | 223 |
| B | 7.3 | 237.7 | 2.5 | 57 | 728 | 195 |
| C | 9.3 | 241.9 | 2.9 | 73 | 801 | 156 |
| E | 19.6 | 128.1 | N.A. | 74 | 402 | 40 |

### Conversion of Amino Saccharide

Amino saccharide (GlcNAc) is converted at elevated temperatures similarly in H₂ environment using Ni/HTO-nMg, Ni/MgO, and Ru/C—NaHCO₃ to study the effects of surface biofunctionality on retro-aldol condensation and the resulting formation of ethanolamine precursor (NAEA). The formation of NAEA was confirmed by analyzing the separated fraction using a nuclear magnetic resonance spectroscopy and mass spectrometry. The ¹H NMR (400 MHz, D₂O) showed the representative chemical shifts of NAEA (1.8 (s, 3H), 3.2 (t, 2H), 3.52 (t, 2H), 8.0 (s, H)), and [M+H]⁺=104 was detected using mass spectrometry with eleetrospray ionization.

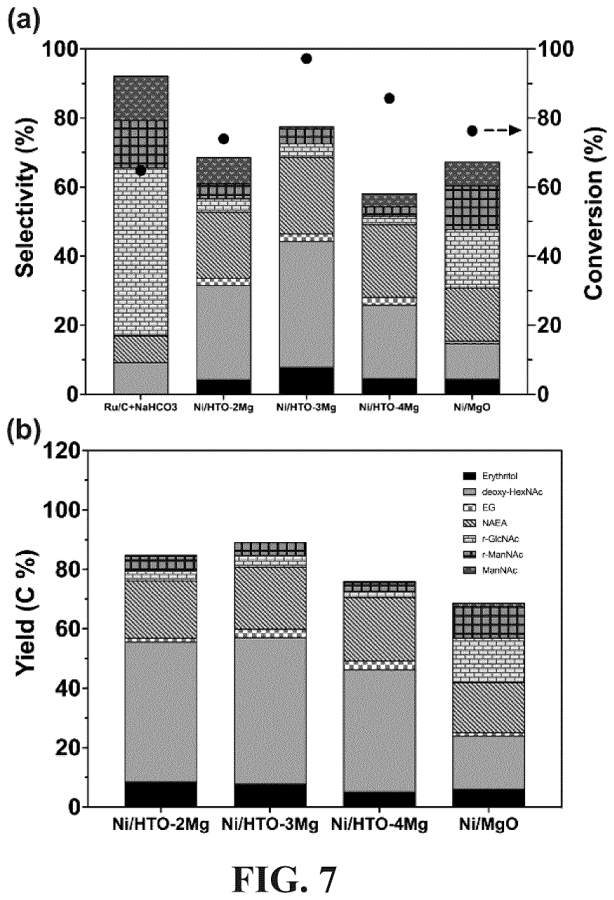

It was found that Ni/HTO-nMg can achieve fairly high NAEA yield at 90° C., which is lower than most of the reported conditions. At 90° C. under 145 psi H₂, Ru/C—NaHCO₃ exhibited the highest selectivity towards r-GlcNAc (48.9%, FIG. 7a) among all tested catalysts, which can be partially attributed to the stronger hydrogenation strength of Ru and its smaller particle size (d_{R}_{ᵤ}=1.8±0.4 nm). Additionally, the weaker basicity of NaHCO₃ leads to slower retro-aldol condensation, thus lowering the selectivity towards NAEA (7.8%). The other identified products are ManNAc, r-ManNAc, and the diastereomers of 2-acetamido-2,3-dideoxyhexitol (expressed as deoxy-HexNAc).

Stark contrast can be seen when comparing the selectivities between Ru/C—NaHCO₃ and Ni/HTO-nMg (FIG. 7a). Ni/HTO-nMg produced little r-GlcNAc and r-ManNAc (selectivities towards both products were below 5%), indicating hydrogenation of GlcNAc and ManNAc became side reaction when using Ni/HTO-nMg. The major components produced by Ni/HTO-nMg were deoxy-HexNAc and NAEA. This result substantiated that retro-aldol condensation as well as dehydration prevail over direct hydrogenation of GlcNAc and ManNAc over Ni/HTO-nMg. The drastic change of reaction pathway can be attributed to basic sites on HTO, which promotes both retro-aldol condensation and dehydration. Since Ni/MgO also has a basic MgO support, its selectivities towards deoxy-HexNAc (10.4%) and NAEA (15.4%) are higher than those of Ru/C—NaHCO₃, but intriguingly, lower than those of Ni/HTO-nMg. This phenomenon is ascribed to the distinct surface characteristics of Ni/MgO and Ni/HTO-nMg. It can be found that the number of surface basic sites of Ni/MgO is significantly smaller than those of Ni/HTO-nMg, which supposedly results from its lower surface area (CO₂-TPD and S_{BET}, Table 1). Moreover, Ni/MgO lacks strong basic sites according to NH₃-TPD analysis (FIG. 5b). With fewer and weaker basic sites on Ni/MgO, retro-aldol condensation and dehydration compete with hydrogenation to a less extent, thus leading to higher selectivities towards r-GlcNAc (17.0%) and r-ManNAc (12.7%). These results conclude that both retro-aldol condensation and dehydration are surface-catalyzed and underscored the importance of basic support that could be adapted for controlling the reaction pathway during GlcNAc conversion.

To maximize the NAEA yield, the reaction was prolonged to 4 h using Ni/HTO-nMg and Ni/MgO to reach nearly full conversion (conversion>95%). Ni/HTO-nMg produced NAEA with yields higher than 19% (FIG. 7b). Particularly, Ni/HTO-4Mg resulted in an NAEA yield of 21%, which is, to the best of our knowledge, the highest NAEA yield obtained at the lowest temperature (90° C.) to date. In comparison, Ni/MgO resulted in a slightly lower NAEA yield (17%) because of more prevalent hydrogenation of GlcNAc and ManNAc. To summarize the reaction pathway, GlcNAc undergoes four parallel reactions at 90° C., including epimerization, direct hydrogenation, dehydration and retro-aldol condensation. The selectivities towards final products were governed by how the later three reactions compete with each other. Direct hydrogenation of GlcNAc is the main route when Ru/C—NaHCO₃ is used, leading to the highest selectivity towards r-GlcNAc. In the presence of solid base (MgO and HTO), both dehydration and retro-aldol condensation are facilitated, thus increasing the selectivities towards NAEA and deoxy-HexNAc whereas hydrogenation of GlcNAc and ManNAc is suppressed.

Additionally, formation rates of deoxy-HexNAc and NAEA during GlcNAc conversion were measured at 60° C. and listed in Table 2.

TABLE 2

| | deoxy-HexNAc | NAEA |
|---|---|---|
| Catalysts | umol/h | (umol/h) |
| A | 4.4 ± 0.2 | 3.9 + 0.4 |
| B | 5.2 ± 0.4 | 5.8 ± 0.4 |
| C | 4.1 ± 0.4 | 5.0 ± 0.8 |
| E | 2.3 ± 0.4 | 2.7 ± 0.4 |

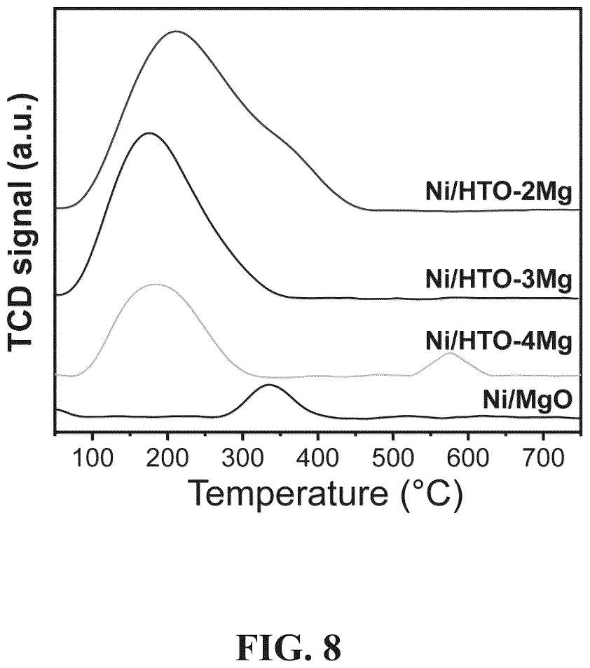

It can be unexpectedly found that the formation of deoxy-HexNAc on Ni/HTO-4Mg is the slower (4.1 umol/h) than on Ni/HTO-2Mg (4.4 umol/h). Ni/HTO-4Mg also resulted in a lower selectivity towards deoxy-HexNAc at 90° C. (FIG. 7a). These results indicate that dehydration is catalyzed by other active species beyond base, considering Ni/HTO-4Mg has the highest number of basic site (Table 1). Surface acid is then tentatively proposed as the additional active site for dehydrating HexNAc because dehydration is typically initiated by protonating hydroxyl group. Accordingly, NH₃-TPD was performed to quantify the acid sites on Ni/HTO-nMg (FIG. 8) and their quantities were found to be inversely correlated with the Mg/Al ratio (Table 1). The increased number of acid site likely results from more exposed Al—OH on the surface when Mg/Al ratio decreases.

It can be concluded that the key to selectively producing NAEA from GlcNAc is promoting retro-aldol condensation while suppressing direct hydrogenation and dehydration. Retro-aldol condensation is spontaneously favored at elevated temperatures provided that the catalyst surface is enriched with basic sites. However, higher temperatures also facilitate dehydration, which competes with retro-aldol condensation and limits the yield of NAEA. Dehydration can be inhibited through reducing the surface acidity by increasing the Mg/Al ratio. With less acid sites, the concerted synergy of hydrogenation and basic sites of Ni/HTO-4Mg led to the highest NAEA yield in this study.

Moreover, the yields of NAEA at 120° C. using catalysts A-D and F-G were listed in Table 3.

TABLE 3

| | Yield_{NAEA} at 120° C. |
|---|---|
| Catalysts | (% of C yield) |
| A | 17.1 |
| B | 21.5 |
| C | 8.6 |
| D | 7.0 |
| F | 1.3 |
| G | 0 |

### CONCLUSION

Ni-doped hydrotalcite-like material can be used as a precursor to synthesize a series of bifunctional Ni/HTO-nMg catalysts, comprising a basic MgAlOₓ support and Ni nanoparticles. At elevated temperatures, the abundant basic sites on HTO allow retro-aldol condensation to outpace direct hydrogenation of GlcNAc, thus achieving an exceptional selectivity towards NAEA. In comparison, Ni/MgO is more selective towards r-GlcNAc because it has fewer surface basic sites. Additionally, dehydration of GlcNAc is identified as another competing reaction, which forms deoxy-HexNAc. The selectivity towards deoxy-HexNAc is inversely correlated with the Mg/Al ratio because dehydration is promoted by not only basic but also acid sites. Therefore, Ni/HTO-4Mg outperforms all tested catalysts, resulting in the highest NAEA yield of 21% at 90° C.

The above examples are intended for illustrating the embodiments of the subject invention and the technical features thereof, but not for restricting the scope of protection of the subject invention. Many other possible modifications and variations can be made without departing from the spirit and scope of the invention as hereinafter claimed. The scope of the subject invention is based on the claims as appended.

What is claimed is:

1. A method for conversion of β-hydroxy carbonyl species, the method comprising:
mixing β-hydroxy carbonyl species with a composite catalyst derived from layered double hydroxides (LDHs), wherein the composite catalyst includes a basic LDHs-derived mixed oxide support and hydrogenation-active species on the basic LDHs-derived mixed oxide support; and
catalyzing retro-aldol condensation and then hydrogenation in hydrogen environment using the composite catalyst to complete conversion of the β-hydroxy carbonyl species.

2. The method of claim 1, wherein the β-hydroxy carbonyl species includes a nitrogen-containing group.

3. The method of claim 1, wherein the β-hydroxy carbonyl species includes a nitrogen-containing group at Ca position.

4. The method of claim 3, wherein the nitrogen-containing group includes an N-acyl-substituted amino moiety.

5. The method of claim 1, wherein the LDHs are doped M³⁺/N²⁺-LDHs, the M³⁺ is a trivalent metal, and the N²⁺ is a bivalent metal.

6. The method of claim 5, wherein the hydrogenation-active species are from doping elements of the doped M³⁺/N²⁺-LDHs, and the total molar quantity of the doping elements and the N²⁺ is 2 to 4 times that of the M³⁺.

7. The method of claim 5, wherein the M³⁺ is Al³⁺, and the N²⁺ is Mg²⁺.

8. The method of claim 1, wherein the hydrogenation-active species are nickel nanoparticles.

9. The method of claim 1, wherein the conversion of the β-hydroxy carbonyl species produces an alcohol compound containing an N-acyl-substituted amino moiety and having fewer carbon atoms than the β-hydroxy carbonyl species.

10. The method of claim 1, wherein the β-hydroxy carbonyl species is from a saccharide.

11. The method of claim 10, wherein the saccharide is monosaccharide.

12. The method of claim 1, wherein the conversion of the β-hydroxy carbonyl species proceeds at a temperature in a range of 60° C. to 120° C.

13. A method for preparing an amino alcohol precursor, comprising:
providing an amino saccharide; and
mixing the amino saccharide with a composite catalyst derived from layered double hydroxides (LDHs) for conversion of the amino saccharide into the amino alcohol precursor by retro-aldol condensation and then hydrogenation under catalysis of the composite catalyst, wherein the composite catalyst includes a basic LDHs-derived mixed oxide support and hydrogenation-active species on the basic LDHs-derived mixed oxide support.

14. The method of claim 13, wherein the amino saccharide includes an N-acyl-substituted amino moiety.

15. The method of claim 13, wherein the LDHs are doped M³⁺/N²⁺ LDHs, the M³⁺ is a trivalent metal, and the N²⁺ is a bivalent metal.

16. The method of claim 15, wherein the hydrogenation-active species are from doping elements of the doped M³⁺/N²⁺-LDHs, and the total molar quantity of the doping elements and the N²⁺ is 2 to 4 times that of the M³⁺.

17. The method of claim 15, wherein the M³⁺ is Al³⁺, and the N²⁺ is Mg²⁺.

18. The method of claim 13, wherein the hydrogenation-active species are nickel nanoparticles.

19. The method of claim 13, wherein amino alcohol precursor is an alcohol compound containing an N-acyl-substituted amino moiety and having fewer carbon atoms than the amino saccharide.

20. The method of claim 13, wherein the amino saccharide is N-acetyl glucosamine.

21. The method of claim 13, wherein the conversion of the amino saccharide into the amino alcohol precursor proceeds in hydrogen environment at a temperature in a range of 60° C. to 120° C.

22. The method of claim 13, wherein a weight ratio between the amino saccharide and the composite catalyst is 2 or less.

23. The method of claim 13, wherein the amino saccharide is N-acetyl glucosamine, and the amino alcohol precursor is N-acetyl ethanolamine.

24. The method of claim 13, wherein the step of providing the amino saccharide includes depolymerizing chitin biomass.
